(19) United States
(12) Patent Application Publication
(10) Pub. No.: US 2024/0336950 A1
(43) Pub. Date: Oct. 10, 2024

(54) Use of a Sustainable, Modified and Enhanced Aquaculture Limulus Amebocyte Lysate Protein for Detection and Characterization of Infectious Pathogens in Biologic Samples for Patient Screening, Diagnosis and Therapeutic Management

(71) Applicants: Rachel Tinker-Kulberg, Hillsborough, NC (US); Terry Earl Brady, The Valley (AI); Anthony Lee Dellinger, Burlington, NC (US); Melinda K. M. Goddard, The Valley (AI)

(72) Inventors: Rachel Tinker-Kulberg, Hillsborough, NC (US); Terry Earl Brady, The Valley (AI); Anthony Lee Dellinger, Burlington, NC (US); Melinda K. M. Goddard, The Valley (AI)

(21) Appl. No.: 18/131,558

(22) Filed: Apr. 6, 2023

(51) Int. Cl. C12Q 1/18

(52) U.S. Cl. CPC C12Q 1/18

(57) ABSTRACT

The present invention represents a breakthrough in addressing the global healthcare challenge of microbial infectious disease diagnosis and management. The invention employs a sustainable, modified, and enhanced protein lysate derived from horseshoe crab aquaculture that allows for rapid, sensitive, and accurate pathogen detection, typing, and determination of antimicrobial susceptibility using biological specimens regardless of opacity, e.g., whole blood. The primary embodiment is highly sensitive to clinically relevant pathogens in complex biological specimens, making it a promising tool for screening, diagnosis and treatment of bloodborne and other microbial infections. The sustainable, modified, and enhanced LAL (smeLAL) substrate and reaction chemistries can provide a cost-effective solution for detecting such pathogens using small volumes of routine hospital and laboratory specimens. The proposed invention also describes a microwell multiplex assay for rapid, culture-free antimicrobial susceptibility testing in biological specimens in less than 5 hours and enables rapid, small specimen volume, and sensitive asymptomatic screening, classification, and antimicrobial susceptibility profiling of diverse pathogens in biological specimens. The invention is both socially and environmentally important and may be used to improve the performance of established diagnostic platforms that detect pathogenic material in biological samples.

### STATEMENT REGARDING FEDERALLY SPONSORED RESEARCH OR DEVELOPMENT

The invention was made with government support under Grant Nos. 1819562, 2101278 and 2212920 awarded by the National Science Foundation. The US government does not have rights to the invention.

### FIELD OF THE INVENTION

The present invention relates to the field of diagnostics and, in particular, a sustainable, modified and enhanced Limulus Amebocyte Lysate (smeLAL) derived from sustainable horseshoe crab and methods thereof which enables rapid screening of asymptomatic patients as well as those with suspected microbial infections by detection of pathogens and for antibiotic susceptibility testing using a small volume of a biological specimen, irrespective of sample opacity.

### BACKGROUND OF THE INVENTION

Sepsis is a major public health and economic concern that results in one death every 2.8 seconds, worldwide [Sepsis Alliance, 2018]. In the United States, sepsis contributes to 270,000 deaths per year [Ferreira et al, 2022], is the leading cost to hospitals [Paoli et al., 2018], and is the primary cause of readmissions [Fingar, 2015]. Globally, sepsis results in 11 million deaths and affects 50 million people each year [Angus et al., 2001; Kumar et al., 2011; Rudd et al., 2020]. Early and accurate detection of pathogens in a biological specimen, prior to onset of sepsis, can mitigate tissue and organ damage, avert mortality, and reduce associated hospital costs [Evans et al., 2021]. Without rapid detection and appropriate antibiotic administration, the risk of patient mortality increases by 7.6% per hour [Kumar et al., 2006].

Despite medical advances, sepsis is often fatal [Angus et al., 2006]. Most who survive it nonetheless suffer from permanent organ damage [Chang et al., 2010], cognitive impairment and/or physical disability [Iwashyna et al., 2010]. The dearth of rapid and accurate diagnostic tests for pathogens in biological specimens has hampered the accuracy and timeliness of clinical decision making, thus propelling sepsis to become the leading cause of premature death worldwide [CDC, 2017]. Lacking timely, accurate detection tools, physicians often rely on indirect assessments of confounding symptoms and clinical pragmatism to initiate treatment within one hour [Buckman et al., 2018]. Consequently, untenable rates of morbidity and mortality persist in a pathogenic era with intensifying risks from antibiotic microbial resistance (AMR) due to unilateral administration of broad-spectrum therapeutics, often compounded by suboptimal drug selection for unknown pathogens and/or patient compliance issues that fail to eradicate otherwise susceptible organisms before resistant mutations occur [Hsu, 2020].

With regard to other prospective detection methods, wild-capture Limulus Amebocyte Lysate (LAL) from horseshoe crabs (Limulus polyphemus) was shown to be the most sensitive (parts-per-trillion), reliable, rapid (1-hour reaction time), and easiest method for bacterial endotoxin testing (BET) by the 1970s. LAL specificity derives from its two distinct Factor C and Factor G pathways. The Factor C pathway is specific to lipopolysaccharide (LPS), unique to the surface of gram-negative bacteria; and the Factor G pathway, which is specific to 1-3-β-D-Glucan (βDG) and represents one of the most abundant cell wall components of pathogenic fungi. Once given approval by the FDA, use of LAL in BET sterility testing for injectable drug and implanted device quality control was widely adopted in industry; whereas constituent complexity and interfering substances in human specimens and inconsistent wild-capture LAL performance precluded its clinical use due to unreliability, as reported in research efforts that spanned decades.

Specifically, two manuscripts in 1970 and 1971 explained interferences in a patient blood sample that affected the LAL assay [Levin et al., 1970 and Levin et al., 1971]. In 1973, Stumacher and researchers at Boston University School of Medicine further described the limitations of wild-capture LAL for detection of endotoxins in human blood [Stumacher et al., 1973]. In 1975, Ronald Elin and colleagues at the NIH published, “A Lack of Clinical Usefulness of LAL for Diagnosis of Endotoxemia” which described the accuracy and reliability limitations of wild-capture LAL for clinical use [Elin et al., 1975]. Numerous reports of assay variability have also described the presence of interfering substances that cause false positives and negatives [Hurley, 1994; Levin, 1970; Levin, 1971], attributed to enzymatic degradation [Rowley et al., 1958; Keene et al., 1961; Skarnes, 1966; Skarnes, 1968] and sequestration by LPS-binding proteins (LPBs; such as alpha-2-macroglobulin [Yoshioka, 1984]). LPS antibodies [Wardle, 1979], high-density lipoproteins [Freudenberg, 1980; Ulevtich, 1981], and antibacterial peptides (BPI, bacterial permeability increasing protein) [Elsbach and Weiss, 1993 and Marra et al. 1994] present in biological specimens [Oroszlam, 1966; Rudbach, 1966].

Various specimen manipulation strategies have been applied and evaluated to overcome biological compatibility issues including: specimen dilution, heating, acidification, digestion or combinations thereof, to enable reliable assay and specimen compatibility [McCabe et al., 1972; Martinez et al., 1973; Stumacher et al., 1973; Feldman and Pearson, 1974; Elin et al., 1975; Zinner and McCabe, 1976; and Gnauck et al., 2015]. In addition to biological inhibitors, anticoagulants, chemical inhibitors and temperature-induced pH changes of the biological specimen have been shown to interfere with the reliability of LAL [Gnauck et al., 2016]. Substrate inconsistency and batch-to-batch variability of pooled LAL reagents derived from wild-capture horseshoe crabs (i.e., ratio and function of the individual protein components) impact precision and compromise further standardization of assay methods, reaction kinetics, and sensitivity [Massignon et al, 1996].

Wild-capture LAL substrate variability has also been linked to many factors, including shifts in protein quality, activity from batch-to-batch, and year-to-year factors associated with environmental circumstances. Wild-capture LAL is collected during the spawning season, when horseshoe crabs are often physically depleted, malnourished, stressed, and exhausted from migration. These issues are compounded by deteriorating environmental conditions, such as warming ocean temperatures and acidification, pollutants and biotic stressors, habitat destruction from coastal development and dredging, and reductions in overall food availability [Wallace et al., 2014; Mattei et al., 2015]. Assessments of horseshoe crab (and many ocean species) reveal wide-ranging population declines in terms of counts, as well as health, mating behavior, movement patterns, and physical size [Chabot and Watson, 2010; Smith et al., 2017, Krisfalusi-Gannon et al., 2018; Mattei et al., 2022].

The industrial process for wild-capture horseshoe crab bleeding and LAL collection is also taxing to animal health and widely considered by scientists and environmental agencies as inhumane [Henry and Wheatly, 1992; Towle and Henry, 2003; Hurton and Berkson, 2006; Allender et al., 2010; Leschen and Correia, 2010; Coates et al., 2012; Anderson et al., 2013]. Despite years of such concerns, daily harvest limits and regulations are still set in cubic feet and based on the total volume of the bed of a typical pickup truck [Flood, 2023]. Wild-capture LAL specimen collection further requires animal transportation to offshore facilities that result in extended ocean habitat extraction, potential exposure to an array of deleterious conditions, and consequent hypoxia for 24-72 hours. The process and its associated stressors drive an estimated mortality rate of some 30% annually [Smith et al., 2020], posing increasing risks of extinction. From a material perspective, wild-capture collection from a weakened and stressed animal can impact LAL substrate quality and consistency. Further disparities arise when combining materials from randomly captured healthy and compromised animals from different regions, amplifying batch-to-batch variability. Wild-capture LAL has nonetheless met industry BET requirements to confirm the sterility of drugs, devices and water. Notably, such testing is typically performed indirectly on pharmaceutical-grade aqueous eluates, rather than the tested source materials, making the process significantly less demanding than assessing complex biological specimens.

As a result, approaches to detect infectious agents in patient samples (e.g., whole blood specimens) continued to include established practices for blood culturing in specific media and growth conditions, which can require exceptionally large specimen volumes often problematic for elderly and pediatric testing. Prior administration of antibiotics can also hinder the culturing objective to grow concentrated and high-density pathogen counts for and species identification. Blood culturing for diagnosis and antimicrobial susceptibility testing (AST) of bacterial infections can require 2 to 7 days [Lamy et al., 2016], minimizing the clinical relevance of the results. This approach also can lead to a delay in appropriate treatment and contribute to the spread of antibiotic-resistant infections [Mancuso et al., 2021]. More recent methods also include targeted, genomic sequencing (i.e., PCR) typically requiring specialized reagents and expertise.

Alternatively, nonspecific, yet faster diagnostic methods leverage indirect assessments, such as phenotypical symptoms, CBC and coagulation assays, as well as tests for procalcitonin, C-reactive protein and lactic acid. However, these indirect methods often fail to provide therapeutic guidance. The microorganisms that lead to sepsis are diverse and complex, with many species and strains that exhibit a wide range of clinical manifestations. The possibility of infections from multiple pathogens can also further complicate patient diagnosis and management. Evolving AMR trends, combined with the spectrum of infectious organisms, underscore the need for novel tools that enable timely detection and effective treatment of sepsis.

In fact, conventional sepsis care pathways rely on empiric administration of broad-spectrum antibiotics in nearly 67% of suspected sepsis patients, whereas use has been found to be warranted in only 13% of cases, significantly contributing to patient organ damage and the rise of AMR pathogens [Rhee et al., 2020]. In practice, antibiotic treatment is often initiated based on patient symptoms and prior to obtaining AST results, typically within 1-3 hours after blood cultures are drawn [Sterling et al., 2015].

Direct detection of infectious agents, as well as early specimen screening, with significant time and cost savings relative to blood culture and PCR, respectively, could therefore address urgent medical needs. Clinical benefits would include appropriate, timely patient antibiotic therapy, institutional safety from pathogen surveillance in healthcare settings, and to slow rising AMR trends. The proposed invention establishes such an assay to detect and categorize pathogens and provide timely therapeutic guidance. By overcoming the barriers to LAL use in biological specimens, the innovation leverages some 450 years of horseshoe crab evolution that enabled the species to survive an onslaught of diverse pathogens [Tanacredi, 2001].

Herein, the term “smeLAL” refers to a sustainable, modified and enhanced LAL substrate derived from aquaculture horseshoe crabs. The term “microbe, microorganism, infectious agent or pathogen” when used with reference to the assay or method refers to gram-negative bacteria or fungi. The term “biological specimen, opaque biological specimen, sample or opaque sample” when used with reference to the assay or methods refers to whole blood, serum, plasma, urine, nasal pharyngeal swabs, semen, sweat, saliva, amniotic fluid, cerebrospinal fluid, gingival fluid, pleural fluid, synovial fluid cyst extract, tissue extracts, ascites fluid, mucus, or similar fluid material derived from a human or other mammal. The term “AST” when used with reference to the assay method refers to inclusion of both antibiotic and antifungal reagents herein.

Notably, smeLAL can detect the presence of free-circulating LPS in patient blood (endotoxemia) in the absence of gram-negative organisms (bacteremia), and LPS is an important diagnostic marker for low-grade systemic inflammation that can be caused by chronic illnesses, such as Crohn's disease, ulcerative colitis, periodontitis, smoker's bronchitis, diverticulitis, obesity, insulin resistance, Type 1 and Type 2 diabetes, hepatitis, non-alcoholic and alcoholic fatty liver disease, chronic kidney disease, atherosclerosis and other cardiometabolic diseases. However, the mean circulating LPS levels in such chronic disease state patients is approximately 10 times lower than those of patients diagnosed with sepsis.

Further, it has been previously described that LAL protein constituents detect the extracellular constituent parts of the pathogens, lipopolysaccharide (LPS) and 1-3-β-D-Glucan (βDG), respectively. The term “protein” when used in reference to smeLAL or wild-capture LAL refers to serine protease zymogens, Factor C, Factor G, Factor B, pro-clotting enzymes, and the precursor protein, coagulogen, that comprise the activated amebocyte lysate coagulation cascade reaction.

When present in a specimen, Factor G serine protease zymogen is activated by βDG present on the surface of fungi. Recognition of βDG triggers activation of Factor G, which binds and initiates the cleavage of proclotting enzymes into clotting enzymes and the hydrolytic conversion of coagulogen into coagulin. The Factor G pathway may be blocked via the addition of the polysaccharide, CM-curdlan, to differentiate LPS and βDG gelation in the coagulation cascade. While extensively used for biomedical sterility testing as mandated by the FDA (BET), the precise kinetics, effect of protein modifications, serine protease zymogen ratios and concentration, as well as differences in proclotting clotting enzyme dynamics remain poorly understood.

### SUMMARY OF THE INVENTION

The proposed invention comprises an aquaculture-derived LAL that is sustainable, modified and enhanced (smeLAL) and testing methods for clinical screening, surveillance and diagnoses, and to perform antimicrobial susceptibility testing (AST) of small volumes of biologic specimens, regardless of opacity. The aquaculture innovations afford the smeLAL greater reactivity than that of wild-capture derived materials [Tinker-Kulberg et al., 2020] for utility in early diagnosis of infection in a clinical specimen prior to the onset of septicemia or sepsis in advance of the release of acute phase reactants and changes in blood pressure or febrile markers. The proposed invention overcomes earlier LAL substrate issues associated with wild-capture reagents for diagnostic use in biological specimens.

The smeLAL and methods described herein yield greater protein density, post-translational protein modifications, allow standardization consistent with respect to reactivity, and provide functionality for clinical endpoint detection of a pathogen in biological specimens. The smeLAL material can be quality controlled to meet clinical reliability standards, maintain batch-to-batch consistency, eliminate wild-capture species impacts, and facilitate scaling for industrial and clinical applications. As such, it would be obvious to someone skilled in the art that smeLAL would be suitable as a replacement of wild-capture LAL in BET.

The performance characteristics of smeLAL in the present invention are principally related to a profound plasticity of native protein expression in domesticated aquaculture horseshoe crabs. In an environment free of documented stressors associated with current wild-capture practices, scientifically engineered nutrition and management, including oxygenation monitoring and optimization, allows for low-impact protein collection from a healthy immunocompetent cohort of captive horseshoe crabs.

With sensitivity to some 70% of the pathogens that lead to sepsis, the smeLAL horseshoe crab blood components, i.e., coagulin, pro-clotting enzymes, Factor C, Factor B and Factor G, are more concentrated, reactive and compatible with biological specimens compared to those isolated from wild-capture horseshoe crabs [Tinker-Kulberg et al., 2020].

In the primary embodiment, the proposed invention comprises smeLAL and a method that enables detection of a pathogenic microorganism in a biological specimen assay without dilution factors, acid treatment, heat treatment, or digestion (application of heat in the presence of an acid) that can otherwise decrease assay sensitivity, reproducibility, reliability and prevent standardization.

The invention can also leverage the in vitro ability to challenge a blood sample to a rubric of known antimicrobials (reactivity and resistance) for AST via the overall presence, absence and level of bactericidal activity in response to a given antimicrobial, obviating the need for empiric broad-spectrum administration. In the primary embodiment, the endpoint reaction is unaffected by specimen opacity and/or the presence of antibiotics or antifungals (administered to the patient prior to specimen collection or utilized in assessing susceptibility), and its gel formation endpoint confirms AST.

Yet another embodiment of the present invention comprises isolation and purification of a biological specimen using conjugated magnetic micro (or nano) spheres or beads and competitive exposure to antimicrobials to rapidly remove interfering and inhibitory biomolecules and proteins from a small specimen volume prior to an smeLAL assay. Pathogens sensitive to the antimicrobial are released by a red blood cell (RBC) carrier and magnetic-affinity bead complex, thus not present to react in the downstream diagnostic assays as a confirmation of antimicrobial susceptibility (e.g., LAL, ELISA, PCR, CRISPR, gram-staining, blood culture or microscopy). Thus, cellular and pathogen capture/affinity creates further novelty when combined with the sensitivity, specificity and reactivity of smeLAL in a small volume screening assay.

### DETAILED DESCRIPTION OF THE INVENTION

The primary embodiment of the invention is related to an smeLAL and method for direct detection of free-circulating LPS and intact gram-negative bacteria or fungi in a small-volume, biological specimen, irrespective of sample opacity. Thus, the invention overcomes issues described in the prior art in the field using LAL substrates that failed to reliably detect the presence of a pathogen in a biological specimen, to differentiate gram-negative bacteria from fungal pathogens, and to determine antimicrobial susceptibility of infectious agents.

In a secondary embodiment of the invention, an smeLAL method for AST using both antibiotic and antifungal susceptibility testing agents is described for pathogen classification, rather than specific identification. In this embodiment, an smeLAL assay method comparing reactions to antibiotic and antifungal reagents differentiates between gram-negative bacteria and fungi. This AST embodiment thus provides a direct determination of pathogen susceptibility using the smeLAL substrate.

The proposed smeLAL invention can be applied a range of biological specimens, including complex samples comprising inhibitors and interferants, without dilution or additive pre-treatment chemistries that can reduce assay sensitivity and reliability. The invention is considered suitable for numerous clinical biological specimens, including but not limited to whole blood, serum, plasma, urine, nasal pharyngeal swabs, semen, sweat, saliva, amniotic fluid, cerebrospinal fluid, gingival fluid, pleural fluid, synovial fluid, cyst extract, tissue extracts, ascites fluid, mucus, etc. irrespective of sample origin, opacity and material preservation conditions (fresh or frozen). Alternatively, the smeLAL invention is further compatible with donor components such as whole blood, plasma, serum, red blood cells, platelets, etc.

Yet another embodiment of the invention encompasses methods for biological specimen preparation suitable for use with commonly used diagnostic platforms to detect pathogenic material. As such, the methods may be incorporated for test formats or devices suitable for the practice of the methods without dilution or manipulation in traditional microbiology assays, such as gram staining, microscopy and blood culture, as well as LAL, ELISA, hemagglutination, PCR and CRISPR assays.

In the primary embodiment, the presence of LPS in the pathway results in the activation of the inactive serine protease zymogen, Factor C, the via Lipid A region of LPS binding to the LPS-binding site and converting phosphorylated or glycosylated Factor C (heavy and light-chain subunits) into activated Factor C (alpha and beta-chain subunits). Activated Factor C nicks the single-chain Factor B zymogen to form Factor B′ (heavy-chain), the glycosylated precursor that is converted to activated Factor B (heavy, alpha and beta-chains). Activated Factor B initiates cleavage of various pro-clotting enzymes to hydrolyze the precursor protein coagulogen into coagulin. Similar to the conversion of fibrinogen into fibrin, the creation of coagulin creates a mesh-like gel or clot network. A Polymixin B or Poly-L-Lysine reagent in the primary embodiment blocks the Factor C pathway by binding to and neutralizing LPS to differentiate LPS and βDG gelation in the coagulation cascade.

The present invention comprises fully characterized, unique LAL composition and co-factors that establish an smeLAL substrate composition when employed with assay methods that overcome reliability and sensitivity issues associated wild-capture LAL when testing biological specimens. In particular, analysis and detection of pathogens in such biological specimens using smeLAL improves sensitivity and specificity by eliminating interferences from obscured detection signals or the need to perform substantial specimen dilutions to remove them, as with wild-capture LAL testing.

The primary embodiment of the present invention employs an smeLAL substrate developed and characterized by sustainable and controlled aquaculture, nutrition, bleeding and specimen processing. The overall amebocyte-driven reactivity of smeLAL is as much as 6-fold greater than that of wild-capture reference LAL at the same total protein concentration, producing a rapid gelation endpoint or “clot.” Critical factors associated with smeLAL performance differ significantly in form and function from wild-capture LAL substrate. Enhancements include protein concentration, composition, function, subunit form and structure, as well as critical co-factor post-translation modifications, and upregulation of pro-clotting enzymes. Substrate reaction kinetics are enhanced by optimized enzymatic protein ratios that catalyze a cascade of reactions to improve overall assay efficiency. Further enhancing smeLAL reactivity are post translational modifications (phosphorylations and glycosylations) to the various serine protein zymogens and enhanced concentrations of the pro-clotting enzymes that characterize the reaction pathway. The distinctive smeLAL composition and associated methods disclosed herein thus provide a compatible and reliable substrate to detect gram-negative bacteria and/or fungi in biological specimens.

In this embodiment of the invention, the smeLAL composition and method establish a process for detecting the presence of a gram-negative bacteria or fungal pathogen and further discriminating between the two in a small volume biological specimen. Two methods comprising either a centrifugation step or functionalized magnetic bead isolation step for a whole blood biological specimen are described herein.

In the embodiment employing a centrifugation method, a pyrogen-free, borosilicate glass container or microtainer is used to collect or transfer from a fresh whole-blood sample in a “red-top” tube (otherwise customarily used for conversion of whole blood to serum and free of coagulant or preservative). An existing patient specimen contained in a collection source comprising the stabilizing and anticoagulation agents sodium citrate, heparin, or EDTA citrate may also be aseptically transferred to the pyrogen-free, borosilicate glass container or microtainer. The specimen is then centrifuged at a minimum of 600 g for 5 minutes at 4° C. to an upper range of 13,000 g for 10 minutes at room temperature (RT) to isolate the RBC fraction. The RBC isolate is then suspended in an endotoxin-free isotonic buffer solution and washed via centrifugation again, as described. The “washed” RBCs are then resuspended and lysed with an endotoxin-free hypotonic buffer solution to equal the original specimen volume. The specimen is further processed for testing using a freeze and thawing cycle at −20° C. to −70° C., or using liquid nitrogen, or another membrane destabilizing process. Analysis is then performed by adding an equal volume (to that of the sample volume) of smeLAL substrate and incubating at 37° C. for 1 hour. The vessel is then gently tilted to 90 and inverted to 180 degrees for visualization of smeLAL-induced gelation (or “clot” formation). The presence or absence of gel clot formation of the sample in the tube indicates pathogen presence or absence, respectively. The specimen preparation and analytical methods for pathogen detection of this embodiment could also be commercially configured and combined for ease of use, as well as adapted to microtiter plate and instrument automation.

In the embodiment employing magnetic bead isolation, functionalized magnetic microbeads or magnetic nanospheres would be added to the biological specimen, obtained as described above, and incubated for a minimum of 5 minutes at RT. The ratio of functionalized magnetic microbeads or nanospheres added to the sample could range from 1:8 up to 1:1 relative to the volume of the sample. The functional groups of the magnetic microbeads or nanospheres would be comprised of benzyl-D-galactopyranoside, CD235a ligands, CD71 ligands, complement components (e.g., C3 and C4), convanavalin A, glycophorins A, B, C, D and E, G-quadruplex DARC aptamer, DNA aptamer, mannose binding lectin, peanut agglutinin, RBC-aptamer 1, RBC-aptamer 2, wheat germ agglutinin, or a combination thereof. Following initial incubation, the sample: bead preparation is then placed in a static magnetic field with a power of 0.1 to 1.0 Tesla for a minimum of 5 minutes. The unbound supernatant is then removed from the magnetically isolated portion of the specimen and beads. The magnetically isolated portion of the specimen may be optionally suspended in an endotoxin-free isotonic buffer solution and washed via centrifugation, as described above. The specimen is lysed with an endotoxin-free hypotonic buffer solution to equal the original specimen volume. The specimen is further processed for testing by a freeze and thawing cycle at −20° C. to −70° C., or using liquid nitrogen, or another membrane destabilizing process. Analysis is then performed by adding an equal volume (to that of the sample volume) of smeLAL substrate and incubating at 37° C. for 1 hour. The vessel is then gently tilted to 90 and inverted to 180 degrees for visualization of smeLAL-induced gelation (or “clot” formation). The specimen preparation method described could also be used for isolation of additional pathogen binding components such as white blood cells and platelets. The specimen preparation and analytical methods for pathogen detection of this embodiment could also be commercially configured and combined for ease of use, as well as adapted to magnetic instrument automation.

Specifically, with respect to automation of the preferred embodiment methods, the “test tube” methods described herein could be optimized for use in microtiter plates of typical configurations (i.e., 48-well, 96-well and 384-well plates) for endpoint measurements with instrumentation. As such, these embodiments could be adapted to existing plate readers and routine hospital and commercial laboratory clinical instruments. Given the reaction kinetics and the presence of magnetic microbeads or nanospheres, analysis with optical, fluorescence, CCD-imaging, ultrasonic, mechanical or electrical impedance techniques could facilitate faster throughput, batch processing, reduce specimen and reagent volume requirements, and simplify assay procedures. Instrumentation may also allow for kinetic assay measurements with the potential for standardization to quantify pathogen concentrations, in addition to pathogen presence, typing, and antibiotic or antifungal susceptibility.

In another embodiment of the proposed invention, diagnostic utility may be enhanced in an smeLAL and antimicrobial (antibiotic and antifungal) susceptibility assay (AST) in the diagnosis of septicemia to control for free-circulating LPS. Inasmuch as LPS is unaffected by antibiotic exposure, the smeLAL AST embodiment could be used to simultaneously assess the antibiotic susceptibility of an infectious agent and differentiate between a bloodstream infection and free-circulating LPS in patients with inflammatory conditions. Notably, the AST embodiment of the proposed invention would be unaffected by variables that have been shown to affect enzymatic and other spectrophotometric assays, e.g., pH, acid-base balance, alkaline phosphatase, bilirubin, C-reactive protein, ferritin, glucose, triglycerides, or other drug molecules often present in biological specimens.

The AST embodiment of the proposed invention entails preparation of triplicate 10-fold serial dilutions of the sample using an endotoxin-free buffer or endotoxin-free whole blood (human or other mammalian sourced). Three specific antibiotic and antifungal agents are then added at minimum inhibitory concentrations (MIC) to each dilution of the three triplicates and incubated for a minimum of 1 to 4 hours at RT to 37° C. The specimen is prepared and processed as described in the centrifugation or functionalized magnetic bead isolation embodiments. Analysis is then performed by adding an equal volume (to that of the sample volume) of smeLAL substrate and incubating at 37° C. for 1 hour. The vessel is then gently tilted to 90 and inverted to 180 degrees for visualization of smeLAL-induced gelation (or “clot” formation). The specimen preparation and analytical methods for pathogen susceptibility testing in this embodiment could also be commercially configured and combined for case of use, as well as adapted to instrument automation.

In the AST embodiment of the invention, antibiotic or antifungal susceptibility testing of any pathogens present in the patient specimen can provide clinical guidance for treating infectious agents if present in a given biological sample. Patients with bloodstream infections typically have less than 100 colony forming units per milliliter [(CFU)/mL] of whole blood, which is consistent with the performance range of the proposed invention. However, analysis of such serial dilutions can enable further discrimination of pathogen classification and susceptibility in cases of exceptionally high pathogen concentrations, as well as generating patterns to estimate bioload in original patient samples. Notably, this inhibition assay approach can also confirm the presence of free-circulating LPS from a loss of gel formation independent of antimicrobial treatment.

Yet another embodiment of the present invention would apply to smeLAL analysis of biological specimens for the detection of bacterial or fungal pathogens, including serum, plasma, urine, nasal pharyngeal swabs, semen, sweat, saliva, amniotic fluid, cerebrospinal fluid, gingival fluid, pleural fluid, synovial fluid cyst extract, tissue extracts, ascites fluid, mucus, etc. Pre-incubation of these biological specimens (fresh or frozen) with a volume of endotoxin-free RBCs would enable smeLAL assay, as well as downstream analysis using other methods, including but not limited to PCR, CRISPR, microbiology blood culture, hemagglutination, microscopy and gram-staining. The methods described would remove contamination and inhibitory factors that can impact sensitive assays, such as ELISA, PCR and CRISPR and/or provide a more concentrated pathogenic specimen suitable for such blood culture, microscopy and gram-staining methods, as well as enable viral detection capabilities.

Wild-capture amebocytes have been derived from the four species of horseshoe crabs: Limulus polyphemus (North American horseshoe crab); Tachypleus tridentatus (Chinese horseshoe crab); Tachypleus gigas (Indo-Pacific horseshoe crab); and Carcinoscorpius rotundicauda (Mangrove horseshoe crab). Each species has distinct amebocyte lysate characteristics, including protein precursors and serine protease zymogen presence and/or concentration likely attributable to exposure to respective pathogens unique to geographical diversity, as well as environmental factors. The aquaculture and use of a sustainable, modified and enhanced amebocyte lysate derived from other horseshoe crab species would nonetheless be expected to achieve similar performance to that used in the primary embodiment of the present invention. Therefore, it would be obvious to one skilled in the art that the analytical performance of a sustainable, modified and enhanced Tachypleus amebocyte lysate (smeTAL) derived from Tachypleus tridentatus or Tacypleus gigas would be consistent with that of the smeLAL substrate of the present invention. Likewise, the use of a sustainable, modified and enhanced Cacrinoscorpisus amebocyte lysate (smeCAL) derived from Carcinoscorpius rotundicauda, would be expected to yield similar performance to that of the smeLAL substrate of the present invention.

Alternatively for each of these embodiments, the inclusion of one or more amebocyte lysates, smeLAL, smeTAL, and/or smeCAL, from aquaculture of two or more of the four species would be capable of blending at collection or processing: cellular phase or lysate phase. In practice, the sustainable, modified and enhanced substrate would be prepared by combining amebocytes optimized with respect to relative reactivities, pH, and buffer stabilized before conversion to lysate. After conversion, sustainable, modified and enhanced lysates could also be blended using optimal ratios of the respective smeLAL, smeTAL and smeCAL with appropriate pH and buffer chemistries. The blended lysate performance from defined ratios would also be anticipated to be at least comparable to that of a single sustainable, modified and enhanced material, such as the smeLAL substrate of the present invention. Assay advantages may also be identified from global diversity with respect to geographic and evolutionary differences of the Limuloid species and the regional pathogens to which they have been exposed. The combination of smeLAL, smeTAL and smeCAL would thus be expected by one skilled in the art to confer as much or more robust assay detection, specificity and sensitivity to a potentially greater pathogen array.

In yet other embodiments, secondary inclusions of non-Limuloid species may be employed to complement the sustainable, modified and enhanced reaction substrate to expand detection capabilities. For example, detection and classification of gram-positive bacteria could be achieved with the addition of a Bombyx mori (silkworm) hemolymph reagent, which precipitates a prophenoloxidase cascade that catalyzes the formation of melanin upon contact with peptidoglycan (PG), resulting in an easily visualized (black) endpoint. A thick PG layer is a distinguishing component of gram-positive bacteria cell walls. Whereby PG is a complex macromolecule composed of long chains of alternating N-acetylglucosamine and N-acetylmuramic acid molecules cross-linked by short peptide chains.

What is claimed:

1. An in vitro method for rapid and simultaneous detection, differentiation and antibiotic or antifungal susceptibility testing of gram-negative bacteria and fungi in a biological specimen, regardless of opacity, from febrile, otherwise symptomatic or asymptomatic individuals using a sustainable, modified and enhanced Limulus amebocyte lysate, comprising the steps:
a. Obtain a biological specimen from a febrile, otherwise symptomatic or asymptomatic individual using sterile techniques;
b. Dilute and incubate the biological specimen with and without a panel of antibiotic and antifungal agents at clinically appropriate concentrations for a predetermined amount of time;
c. Purify the biological specimen to isolate a blood cell fraction, or in the absence of red blood cells in the biological specimen, adding and incubating the biological specimen with sterile whole blood and purifying the biological specimen to isolate a red blood cell fraction;
d. Resuspend and wash the biological specimen red cells in an endotoxin-free isotonic buffer;
e. Lyse the biological specimen (or reagent) red cells with endotoxin-free hypotonic buffer and destabilize using a freeze and thawing cycle;
f. Add sustainable, modified and enhanced Limulus amebocyte lysate to the washed and lysed biological specimen and incubate for a period from 30 to 120 minutes at 37° C.;
g. Visually assess the viscosity of the biological specimen and sustainable, modified and enhanced Limulus amebocyte lysate endpoint by tilting and inverting the vessel, or by using an optical, fluorescent, ultrasonic, mechanical and electrical impedance or another such instrument;
wherein the formation of a gel-like fluid clot and a change in the fluid dynamics of the gel-like fluid clot in the biological specimen relative to a reference or control sample indicates that gram-negative bacteria and/or fungi are present and, if so, susceptible to an antimicrobial agent.

2. The method of claim 1, wherein the antibiotic or antifungal susceptibility testing of gram-negative bacteria or fungi is determined in less than 5 hours.

3. The method of claim 1, wherein the antibiotic or antifungal susceptibility testing detects and determines the antimicrobial susceptibility of gram-negative bacteria or fungi and identifies an effective treatment without needing to determine the gram-negative bacteria or fungi species or strains.

4. The method of claim 1, wherein the antibiotic or antifungal susceptibility testing does not require culture or amplification of the gram-negative bacteria or fungi prior to an antimicrobial susceptibility challenge.

5. The method of claim 1, wherein the biological specimen is whole blood, serum, plasma, urine, nasal pharyngeal swabs, semen, sweat, saliva, amniotic fluid, cerebrospinal fluid, gingival fluid, pleural fluid, synovial fluid, cyst extract, tissue extracts, ascites fluid, or mucus.

6. The method of claim 1, wherein the biological specimen volume is less than 1 milliliter.

7. The method of claim 1, wherein the sustainable, modified and enhanced Limulus amebocyte lysate consists concentrated and modified serine protein zymogens and protein precursors that demonstrate up to 6-fold greater reactivity compared to material derived from wild-capture sources.

8. The method of claim 1, wherein the biological specimen is diluted using serial dilutions.

9. The method of claim 1, wherein free-circulating LPS is differentiated from gram-negative bacteria in the biological specimen.

10. The method of claim 1, wherein the biological specimen is purified using centrifugation between 1,300-4,100 g for 5-10 minutes at 4° C. to RT conditions.

11. The method of claim 1, wherein the biological specimen is purified using functionalized magnetic microbeads or functionalized magnetic nanospheres containing one or more functional groups comprised of: benzyl-D-galactopyranoside, CD235a ligands, CD71 ligands, complement components (e.g., C3 and C4), convanavalin A, glycophorins A, B, C, D and E, G-quadruplex DARC aptamer, DNA aptamer, mannose binding lectin, peanut agglutinin, RBC-aptamer 1, RBC-aptamer 2, wheat germ agglutinin, or a combination thereof and placing the biological specimen in a 0.1 to 1.0 Tesla magnetic field.

12. The method of claim 1, wherein the detection of gram-negative bacteria or fungi in the biological specimen is determined by the formation of a gel-like clot after incubating the biological specimen with sustainable, modified and enhanced Limulus amebocyte lysate for a period of 30 to 120 minutes at 37° C.

13. The method of claim 1, wherein the antibiotic and antifungal susceptibility testing of the gram-negative bacteria or fungi in the biological specimen is determined by reduced and blocked formation of a gel-like clot after incubating the opaque biological specimen with sustainable, modified and enhanced Limulus amebocyte lysate for a period of 30 to 120 minutes at or around 37° C. compared to the control.

14. The method of claim 1, wherein a volume of endotoxin-free whole blood is added the biological specimen and incubated for a predetermined period of time.

15. The method of claim 1, wherein the reagents can be kit configured for commercial use.

16. The method of claim 1, wherein the reagents can be automated using instrumentation.

17. The method of claim 1, wherein the biological specimen may be used for PCR, CRISPR, hemagglutination, ELISA, microbiology blood culture, microscopy and gram-staining, and/or other alternative downstream analytical techniques.

18. The method of claim 1, wherein the sustainable, modified and enhanced Limulus amebocyte lysate further comprises sustainable, modified and enhanced Tachypleus amebocyte lysate, sustainable, modified and enhanced Cacrinoscorpisus amebocyte lysate, or combinations thereof.

19. The method of claim 1, wherein the sustainable, modified and enhanced Limulus amebocyte lysate further comprises Bombyx mori (silkworm) hemolymph for detection of gram-positive bacteria.

20. A kit for detecting the presence and determining the antimicrobial susceptibility and susceptibility of gram-negative bacteria or fungi in a biological sample in less than 5 hours, the kit comprising: (a) an endotoxin-free vial or other vessel containing sustainable, modified and enhanced Limulus amebocyte lysate, (b) an endotoxin-free vial or other vessel containing functionalized magnetic microbeads or nanospheres with one or more functional groups, (c) an endotoxin-free isotonic buffer solution supplemented with 15 mM sodium chloride, (d) an endotoxin-free hypotonic buffer solution supplemented with 2 mM calcium chloride and 2 mM magnesium chloride, (d) multiple vials or other vessels containing endotoxin-free antimicrobial reagents and (e) a standardized endotoxin-free diluent comprising either human or other mammalian whole blood source.
